(19) United States
(12) Patent Application Publication
(10) Pub. No.: US 2025/0235504 A1
(43) Pub. Date: Jul. 24, 2025

(54) ENHANCEMENT OF CD47 BLOCKADE WITH TAXANES FOR CD47+ CANCER THERAPY

(71) Applicant: Pfizer Inc., New York, NY (US)

(72) Inventors: Christina Lindsay KIRKHAM, Ottawa, Ontario (CA); Gloria Hoi Ying LIN, Oakville, Ontario (CA); Emma Cecilia LINDEROTH, Toronto, Ontario (CA); Penka Slavcheva SLAVOVA-PETROVA, Toronto, Ontario (CA); Robert Adam UGER, Richmond Hill (CA); Natasja Nielsen VILLER, Etobicoke, Ontario (CA)

(73) Assignee: Pfizer Inc., New York, NY (US)

(21) Appl. No.: 18/705,445

(22) PCT Filed: Oct. 26, 2022
(86) PCT No.: PCT/IB2022/060295

§ 371 Date: Apr. 26, 2024

Related U.S. Application Data
(60) Provisional application No. 63/273,851, filed on Oct. 29, 2021.

(51) Int. Cl. A61K 38/17; A61K 31/337; A61P 35/00

(52) U.S. Cl. CPC A61K 38/1774; A61K 31/337; A61P 35/00

(57) ABSTRACT

The invention provides combinations of materials and methods for disease treatment, such as CD47+ cancer treatment. The combinations include a CD47 blocking agent and a taxane.

### FIELD

This invention relates to methods of using an agent that blocks the CD47/SIRPα interaction. More particularly, the invention relates to methods and means that, in combination, are useful for improving cancer therapy.

### BACKGROUND

Cancer cells can be targeted for destruction by antibodies that bind to cancer cell antigens, and through recruitment and activation of macrophages by way of Fc receptor binding to the Fc portion of that antibody. Binding between CD47 on cancer cells and SIRPα on macrophages transmits a “don't eat me” signal that enables many tumour cells to escape destruction by macrophages. It has been shown that inhibition of the CD47/SIRPα interaction (CD47 blockade) will allow macrophages to “see” and destroy the target CD47+ cancer cell. The use of SIRPα to treat cancer by CD47 blockade is described in international Patent Publication No. WO 2010/130053.

In International Patent Publication No. WO 2014/094122, a protein agent that inhibits the interaction between CD47 and SIRPα is described. This CD47 blocking agent is a form of human SIRPα that binds CD47, and incorporates a particular region of its extracellular domain linked with a particularly useful form of an IgG1-based Fc region (TTI-621), or an IgG4-based Fc region (TTI-622). In this form, the SIRPαFc agent shows dramatic effects on the viability of cancer cells that present with a CD47+ phenotype. The effect is seen particularly on acute myelogenous leukemia (AML) cells, and on many other types of cancer including both liquid (blood) and solid forms including cancer, lung and ovarian cancer. A soluble form of SIRPα having significantly altered primary structure and enhanced CD47 binding affinity is described in WO2013/109752. Still other useful forms of SIRPα include those that are bispecific and have active anti-cancer proteins fused therewith.

Other CD47 blocking agents have been described in the literature and these include various CD47 antibodies [see for instance U.S. Pat. No. 8,562,997 (Stanford), and international Patent Publication No. WO 2014/123580 (InhibRx′)], each comprising different antigen binding sites but having, in common, the ability to compete with endogenous SIRPα for binding to CD47, thereby to allow interaction with macrophages and, ultimately, to increase the rate of CD47+ cancer cell depletion. These CD47 antibodies have activities in vivo that are quite different from those intrinsic to SIRPα-based agents. The latter, for instance, display negligible binding to red blood cells whereas the opposite property in CD47 antibodies creates a need for strategies that accommodate the agent “sink” that follows administration.

Still other agents are proposed for use in blocking the CD47/SIRPα axis. These include CD47Fc proteins (see Viral Logic's WO2010/083253), and SIRPα antibodies as described in UHN's WO2013/056352, Stanford's WO2016/022971, Eberhard's U.S. Pat. No. 6,913,894, and elsewhere.

The CD47 blockade approach in anti-cancer agent development shows great promise. It would be useful to provide methods and means for improving the effect of these agents, and in particular for improving the effect of the CD47 blocking agents, especially those that incorporate SIRPα.

### SUMMARY

In some embodiments, it is shown herein that the anti-cancer effect of a CD47 blocking agent can be improved when combined with a taxane such as paclitaxel. More particularly, significant improvement in cancer cell depletion is seen when CD47+ cancer cells are treated with a CD47 blocking agent, such as a SIRPα-based agent, in combination with a taxane. The two agents synergize in their effects on cancer cells, and result in the depletion of more cancer cells than can be accounted for by the sum of their individual effects. The results indicate that the taxane does not itself trigger the phagocytosis responsible for cancer cell depletion, but it does enhance this effect of the CD47 blocking agent.

In one aspect, there is provided a method for treating a subject presenting with CD47+ cancer cells, comprising administering a treatment-effective agent combination comprising a CD47-binding form of SIRPα and a taxane such as paclitaxel.

In a related aspect, there is provided the use of a SIRPα-based agent in combination with a taxane for the treatment of a subject presenting with CD47+ cancer.

There is also provided, in another aspect, a combination of anti-cancer agents comprising a SIRPα-based CD47 blocking agent and a taxane, together with instructions teaching their use in the treatment method herein described.

In preferred embodiments, the CD47 blocking agent is a SIRPαFc and most preferably is either TTI-621 of SEQ ID NO: 3 or TTI-622 of SEQ ID NO: 9.

To the extent that embodiments, details, or variations are described herein with reference to one particular SIRPα-based drug or taxane, it should be understood that the same embodiments, details, and variations are intended to apply to others identified herein, unless this document or context explicitly indicates otherwise.

Various details and aspects are described herein as treating or methods of treating. In all such circumstances, it should be understood that related or equivalent aspects include the materials/compositions described herein for use in treatment; and the materials/compositions described herein for use in the manufacture of medicaments for treatment of diseases or conditions described herein.

The headings herein are for the convenience of the reader and not intended to be limiting. Other aspects of the invention will be apparent from the detailed description and claims that follow.

Additional embodiments (E) of the invention are summarized in the following numbered paragraphs:
E1. A method for treating a subject presenting with CD47+ cancer cells, comprising administering to the subject a CD47 blocking agent, and a taxane.
E2. A method for improving the treatment of a subject presenting with CD47+ cancer cells, said subject being treated with a CD47 blocking agent, the method comprising administering to the subject a taxane.
E3. A method for improving the treatment of a subject presenting with CD47+ cancer cells, said subject being treated with a taxane, the method comprising administering to the subject a CD47 blocking agent.
E4. A CD47 blocking agent and a taxane for use in combination to treat a subject with CD47+ cancer cells.
E5. A taxane for use in combination with a CD47 blocking agent to treat a subject presenting with CD47+ cancer cells.
E6. Use of a CD47 blocking agent in the manufacture of a medicament for use in combination with a taxane for the treatment of cancer in a subject presenting with CD47+ cancer cells.
E7. Use of a taxane in the manufacture of a medicament for use in combination with a CD47 blocking agent for the treatment of cancer in a subject presenting with CD47+ cancer cells.
E8. The method, use, or drug-for-use according to any one of E1-E7, wherein the taxane is selected from docetaxel and paclitaxel.
E9. The method, use, or drug-for-use according to E8, wherein the taxane is paclitaxel.
E10. The method, use, or drug-for-use according to any one of E1-E9, wherein the CD47 blocking agent comprises a CD47-binding form of human SIRPα.
E11. The method, use, or drug-for-use according to E10, wherein the CD47-binding form of human SIRPα is a CD47-binding fragment of human SIRPα.
E12. The method, use, or drug-for-use according to E11, wherein the CD47 binding fragment of human SIRPα comprises the IgV domain of human SIRPα.
E13. The method, use, or drug-for-use according to any one of E10-E12, wherein the CD47 blocking agent comprises the CD47-binding form of human SIRPα attached to a heterologous peptide or protein.
E14. The method, use, or drug-for-use according to any one of E1-E13, wherein the CD47 blocking agent is an Fc fusion protein comprising the IgV domain of soluble human SIRPα variant 2 attached to an antibody constant region (Fc).
E15. The method, use, or drug-for-use according to E14, wherein the Fc fusion protein comprising soluble SIRPα comprises SEQ ID NO: 1.
E16 The method, use, or drug-for-use according to E7, wherein the Fc fusion protein comprising soluble SIRPα comprises SEQ ID NO: 2.
E17. The method, use, or drug-for-use according to any one of E1-E3, wherein the CD47 blocking agent comprises soluble SIRPα having one or more amino acid substitutions selected from L4V/I, V6I/L, A21V, V27I/L, 131T/S/F, Q37W, Q37H, E47V/L, K53R, E54Q, E54P, H56P/R, S66T/G, K68R, M72R, V92I, F94V/L, V63I, and F103V.
E18. The method, use, or drug-for-use according to any one of E1-E17, wherein the subject presents with CD47+ cancer cells that are blood cancer cells or solid tumour cells.
E19. The method, use, or drug-for-use according to any one of E1-E17, wherein the subject presents with CD47+ cancer cells that are blood cancer cells.
E20. The method, use, or drug-for-use according to E19, wherein the blood cancer cells are leukemia cells.
E21. The method, use, or drug-for-use according to E13, wherein the leukemia cells are acute lymphocytic leukemia (ALL) cells; acute myeloid leukemia (AML) cells; chronic lymphocytic leukemia (CLL) cells; chronic myelogenous leukemia (CML) cells; myeloproliferative disorder/neoplasm (MPDS) cells; or myelodysplastic syndrome cells.
E22. The method, use, or drug-for-use according to E19, wherein the blood cancer cells are lymphoma cells.
E23. The method, use, or drug-for-use according to E22, wherein the lymphoma is Hodgkin's lymphoma, indolent non-Hodgkin's lymphoma, aggressive non-Hodgkin's lymphoma, Burkitt's lymphoma, small cell and follicular lymphoma, or large cell follicular lymphoma.
E24. The method, use, or drug-for-use according to E19, wherein the blood cancer is a myeloma.
E25. The method, use, or drug-for-use according to E24, wherein the myeloma is multiple myeloma (MM), giant cell myeloma, heavy-chain myeloma, or light chain or Bence-Jones myeloma.
E26. The method, use, or drug-for-use according to E18, wherein the cancer cells comprise solid tumour cells.
E27. The method, use, or drug-for-use according to E26, wherein the solid tumour cancer cells are breast cancer cells.
E28. The method, use, or drug-for-use according to E26, where in the solid tumour cancer cells are lung cancer cells.
E29. The method, use, or drug-for-use according to E26, wherein the solid tumour cancer cells are ovarian cancer cells.
E30. A combination of anti-cancer agents, comprising an amount of a CD47 blocking agent effective to deplete CD47+ cancer cells, and an amount of paclitaxel effective to enhance depletion of CD47+ cancer cells, together with instructions teaching the use thereof according to E1-E22.
E31. The use of the combination according to E30, for the treatment of a subject presenting with CD47+ disease cells.
E32. The use according to E31, wherein the CD47+ disease cells are CD47+ cancer cells.
E33. The use according to E32, wherein the CD47+ cancer cells comprise blood cancer cells or solid tumour cells.
E34. The use according to E33, wherein the cancer cells are cells of a cancer type selected from acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome.
E35. The use according to E33, wherein the cancer is a lymphoma selected from a Hodgkin's lymphoma, indolent non-Hodgkin's lymphoma, aggressive non-Hodgkin's lymphoma, Burkitt's lymphoma, small cell follicular lymphoma, and large cell follicular lymphoma.
E36. The use according to E33, wherein the cancer is a myeloma selected from multiple myeloma (MM), giant cell myeloma, heavy-chain myeloma, and light chain or Bence-Jones myeloma.
E37. A kit comprising unit dose formulations of a CD47 blockade drug and a taxane.
E38. The kit according to E37, wherein the CD47 blockade drug and the taxane are packaged together but not in admixture.

These and other aspects of the invention are now described in greater detail with reference to the accompanying drawings, in which:

### BRIEF REFERENCE TO THE DRAWINGS

FIG. 1 demonstrates that when monocyte-derived macrophages are cultured in the presence of the paclitaxel there is an increase in SIRPαFc-induced phagocytosis of target cells versus untreated macrophages, and

FIG. 2A and FIG. 2B demonstrate that when monocyte-derived macrophages and target cells are both cultured in the presence of paclitaxel there is an increase in TTI-621-induced or TTI-622-induced phagocytosis versus macrophages and target cells that were not cultured in the presence of paclitaxel.

### DETAILED DESCRIPTION

The present invention provides an improved method for treating subjects that present with cancer cells and tumours, and other disease cells, that have a CD47+ phenotype. In this method, subjects receive a combination of a CD47 blocking agent that is a CD47-binding form of SIRPα, and a taxane. In combination, the anti-cancer effect of this combination is superior to the effects of either agent alone or of in the addition of the effect of each agent as a monotherapy. The synergism is believed to result particularly when the CD47 blocking agent is a soluble SIRPαFc-based agent.

Thus, the present treatment method combines a CD47-binding form of SIRPα, as a CD47 blocking agent, and a taxane. A CD47 blocking agent is defined herein as a CD47-binding agent that interferes with and dampens signal transmission that results when CD47 interacts with macrophage-presented SIRPα. CD47-binding forms of human SIRPα are the preferred CD47 blocking agents for use in the combination herein disclosed. These agents are based on the extracellular region of human SIRPα. They comprise at least a part of the extracellular region sufficient to confer effective CD47 binding affinity and specificity. So-called “soluble” forms of SIRPα, lacking the membrane anchoring and intracellular components, are described in the literature and include those referenced in WO 2010/070047 (Novartis), WO2013/109752 (Stanford), and WO2014/094122 (Trillium).

In a preferred embodiment, the soluble form of SIRPα is an Fc fusion. More particularly, the agent suitably comprises a CD47-binding fragment of human SIRPα protein, in a form fused directly, or indirectly, with an antibody constant region, or Fc (fragment crystallisable). Unless otherwise stated, the term “human SIRPα” as used herein refers to a wild type, endogenous, mature form of human SIRPα. In humans, the SIRPα protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 80% of the forms of SIRPα present in humans, and both are embraced herein by the term “human SIRPα”. Also embraced by the term “human SIRPα” are the minor forms thereof that are endogenous to humans and have the same property of triggering signal transduction through CD47 upon binding thereto. The present invention is directed most particularly to the agent combinations that include the human SIRP variant 2 form, or V2.

In the present agent combination, useful SIRPαFc fusion proteins comprise one of the three so-called immunoglobulin (Ig) domains that lie within the extracellular region of human SIRPα. More particularly, the present SIRPαFc proteins incorporate residues 32-137 of human SIRPα (a 106-mer), which constitute and define the IgV domain of the V2 form according to current nomenclature. This SIRPα sequence, shown below, is referenced herein as SEQ ID NO: 1.

(SEQ ID NO: 1)
EELQVIQPDKSVSVAAGESAILHCTVTSLIPVGPIQWERGAGPARELIY
NQKEGHFPRVTTVSESTKRENMDFSISISNITPADAGTYYCVKFRKGSP
DTEFKSGA

In a preferred embodiment, the SIRPαFc fusion proteins incorporate the IgV domain as defined by SEQ ID NO: 1, and additional, flanking residues contiguous within the SIRPα sequence. This preferred form of the IgV domain, represented by residues 31-148 of the V2 form of human SIRPα, is a 118-mer having SEQ ID NO: 6 shown below:

(SEQ ID NO: 6)
EEELQVIQPDKSVSVAAGESAILHCTVTSLIPVGPIQWERGAGPARELI
YNQKEGHFPRVTTVSESTKRENMDFSISISNITPADAGTYYCVKFRKGS
PDTEFKSGAGTELSVRAKPS

The present SIRPα fusion proteins can also incorporate an Fc region having effector function. Fc refers to “fragment crystallisable” and represents the constant region of an antibody comprised principally of the heavy chain constant region and components within the hinge region. Suitable Fc components thus are those having effector function. An Fc component “having effector function” is an Fc component having at least some effector function, such as at least some contribution to antibody-dependent cellular cytotoxicity or some ability to fix complement. Also, the Fc will at least bind to Fc receptors. These properties can be revealed using assays established for this purpose. Functional assays include the standard chromium release assay that detects target cell lysis. By this definition, an Fc region that is wild type IgG1 or IgG4 has effector function, whereas the Fc region of a human IgG4 mutated to eliminate effector function, such as by incorporation of an alteration series that includes Pro233, Val234, Ala235 and deletion of Gly236 (EU), is considered not to have effector function. In a preferred embodiment, the Fc is based on human antibodies of the IgG1 isotype. The Fc region of these antibodies will be readily identifiable to those skilled in the art. In embodiments, the Fc region includes the lower hinge-CH2-CH3 domains.

In a specific embodiment, the Fc region is based on the amino acid sequence of a human IgG1 set out as P01857 in UniProtKB/Swiss-Prot, residues 104-330, and has the amino acid sequence shown below and referenced herein as SEQ ID NO: 2:

(SEQ ID NO: 2)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK*

Thus, in embodiments, the Fc region has either a wild type or consensus sequence of an IgG1 constant region. In alternative embodiments, the Fc region incorporated in the fusion protein is derived from any IgG1 antibody having a typical effector-active constant region. The sequences of such Fc regions can correspond, for example, with the Fc regions of any of the following IgG1 sequences (all referenced from GenBank), for example: BAG65283 (residues 242-473), BAC04226.1 (residues 247-478), BAC05014.1 (residues 240-471), CAC20454.1 (residues 99-320), BAC05016.1 (residues 238-469), BAC85350.1 (residues 243-474), BAC85529.1 (residues 244-475), and BAC85429.1 (residues (238-469).

In other embodiments, the Fc region has a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank.

In a specific embodiment, the Fc region is based on the amino acid sequence of a human IgG4 set out as P01861 in UniProtKB/Swiss-Prot, residues 99-327, and has the amino acid sequence shown below and referenced herein as SEQ ID NO: 7:

(SEQ ID NO: 7)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

In embodiments, the Fc region incorporates one or more alterations, usually not more than about 10, e.g., up to 5 such alterations, including amino acid substitutions that affect certain Fc properties. In one specific and preferred embodiment, the Fc region incorporates an alteration at position 228 (EU numbering), in which the serine at this position is substituted by a proline (S²²⁸P), thereby to stabilize the disulfide linkage within the Fc dimer. Other alterations within the Fc region can include substitutions that alter glycosylation, such as substitution of Asn²⁹⁷ by glycine or alanine; half-life enhancing alterations such as T²⁵²L, T²⁵³S, and T²⁵⁶F as taught in U.S. Pat. No. 62,777,375, and many others. Particularly useful are those alterations that enhance Fc properties while remaining silent with respect to conformation, e.g., retaining Fc receptor binding.

In a specific embodiment, and in the case where the Fc component is an IgG4 Fc, the Fc incorporates at least the S228P mutation, and has the amino acid sequence set out below and referenced herein as SEQ ID NO: 8:

(SEQ ID NO: 8)
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

The CD47 blocking agent used in the combination is thus preferably a SIRP fusion protein useful to inhibit the binding of human SIRPα and human CD47, thereby to inhibit or reduce transmission of the signal mediated via SIRPα-bound CD47, the fusion protein comprising a human SIRPα component and, fused therewith, an Fc component, wherein the SIRPα component comprises or consists of a single IgV domain of human SIRPα V2 and the Fc component is the constant region of a human IgG having effector function.

In one embodiment, the fusion protein comprises a SIRPα component consisting at least of residues 32-137 of the V2 form of wild type human SIRPα, i.e., SEQ ID NO: 1. In a preferred embodiment, the SIRPα component consists of residues 31-148 of the V2 form of human SIRPα, i.e., SEQ ID NO: 6. In another embodiment, the Fc component is the Fc component of the human IgG1 designated P01857, and in a specific embodiment has the amino acid sequence that incorporates the lower hinge-CH2-CH3 region thereof i.e., SEQ ID NO: 2.

In a preferred embodiment, therefore, the SIRPαFc fusion protein is provided and used in a secreted dimeric fusion form, wherein the fusion protein incorporates a SIRPα component having SEQ ID NO: 1 and preferably SEQ ID NO: 6 and, fused therewith, an Fc region having effector function and having SEQ ID NO: 2 or SEQ ID NO: 8.

When the SIRPα component is SEQ ID NO: 6, this fusion protein comprises SEQ ID NO: 3, shown below:

(SEQ ID NO: 3)
EEELQVIQPDKSVSVAAGESAILHCTVTSLIPVGPIQWERGAGPARELI
YNQKEGHFPRVTTVSESTKRENMDFSISISNITPADAGTYYCVKFRKGS
PDTEFKSGAGTELSVRAKPSDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
GK

In alternative embodiments, the Fc component of the fusion protein is based on an IgG4, and preferably an IgG4 that incorporates the S²²⁸P mutation. In the case where the fusion protein incorporates the preferred SIRPα IgV domain of SEQ ID NO: 6, the resulting IgG4-based SIRPα-Fc protein has SEQ ID NO: 9, shown below:

(SEQ ID NO: 9)
EEELQVIQPDKSVSVAAGESAILHCTVTSLIPVGPIQWERGAGPARELI
YNQKEGHFPRVTTVSESTKRENMDFSISISNITPADAGTYYCVKFRKGS
PDTEFKSGAGTELSVRAKPSESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK

In a preferred embodiment, the fusion protein comprises, as the SIRPα IgV domain of the fusion protein, a sequence that is SEQ ID NO: 6. The preferred SIRPαFc is SEQ ID NO: 3.

The SIRPα sequence incorporated within the CD47 blocking agent can be varied, as described in the literature. That is, useful substitutions within SIRPα include one or more of the following: L⁴V/I, V⁶I/L, A²¹V, V²⁷I/L, I³¹T/S/F, Q³⁷W, Q³⁷H, E⁴⁷V/L, K⁵³R, E⁵⁴Q, E⁵⁴P, H⁵⁶P/R, S⁶⁶T/G, K⁶⁸R, M⁷²R, V⁹²I, F⁹⁴V/L, V⁶³I, and/or F¹⁰³V. (Amino acid position numbers correspond with sequences shown herein, including SEQ ID NO: 6). Substitutions that remove glycosylation sites are also acceptable. Suitable variants will display adequate CD47-binding and antagonist activity, with respect to signal transmission between CD47 and SIRPα.

In the SIRPαFc fusion protein, the SIRPα component and the Fc component are fused, either directly or indirectly, to provide a single chain polypeptide that is ultimately produced as a dimer in which the single chain polypeptides are coupled through intrachain disulfide bonds formed within the Fc region. The nature of the fusing region is not critical. The fusion may be direct between the two components, with the SIRP component constituting the N-terminal end of the fusion and the Fc component constituting the C-terminal end. Alternatively, the fusion may be indirect, through a linker comprised of one or more amino acids, desirably genetically encoded amino acids, such as two, three, four, five, six, seven, eight, nine or ten amino acids, or any number of amino acids between 5 and 100 amino acids, such as between 5 and 50, 5 and 30 or 5 and 20 amino acids. A linker may comprise a peptide that is encoded by DNA constituting a restriction site, such as a BamHI, ClaI, EcoRI, HindIII, PstI, SalI and XhoI site and the like.

The linker amino acids typically and desirably have some flexibility to allow the Fc and the SIRP components to adopt their active conformations. Residues that allow for such flexibility typically are Gly, Asn and Ser, so that virtually any combination of these residues (and particularly Gly and Ser) within a linker is likely to provide the desired linking effect. In one example, such a linker is based on the so-called G₄S sequence (Gly-Gly-Gly-Gly-Ser) (SEQ ID NO: 4) which may repeat as (G₄S)ₙ where n is 1, 2, 3 or more, or is based on (Gly)n, (Ser)n, (Ser-Gly)n or (Gly-Ser)n and the like. In another embodiment, the linker is GTELSVRAKPS (SEQ ID NO: 5). This sequence constitutes SIRPα sequence that C-terminally flanks the IgV domain (it being understood that this flanking sequence could be considered either a linker or a different form of the IgV domain when coupled with the IgV minimal sequence described above). It is necessary only that the fusing region or linker permits the components to adopt their active conformations, and this can be achieved by any form of linker useful in the art.

The SIRPαFc can, as a single chain polypeptide, be fused with a different Fc fusion protein to provide an Fc dimer that is bispecific in its affinity. For instance, SIRPαFc can be coupled with an Fc fusion or antibody that binds a tumour-specific antigen, such as epidermal growth factor receptor (EGFR) other target cancer cell antigens. Bispecific proteins can also be generated by coupling/fusing a protein of medical interest to the N- or C-terminus of SIRPα or SIRPαFc.

As noted, the SIRPαFc fusion is useful to inhibit interaction between SIRPα and CD47, thereby to block signalling across this axis. Stimulation of SIRPα on macrophages by CD47 is known to inhibit macrophage-mediated phagocytosis by deactivating myosin-II and the contractile cytoskeletal activity involved in pulling a target into a macrophage. Activation of this cascade is therefore important for the survival of CD47+ disease cells, and blocking this pathway enables macrophages to eradicate or at least reduce the vitality or size of the CD47+ disease cell population. A CD47 blocking agent thus can be any agent that achieves this, including SIRPα and SIRPαFc, a CD47 antibody and bispecific forms thereof, as well as a CD47Fc fusion or a SIRPα antibody. These can referred to collectively as anti-CD47 agents.

The term “CD47+” is used with reference to the phenotype of cells targeted for binding by the present polypeptide agents. Cells that are CD47+ can be identified by flow cytometry using CD47 antibody as the affinity ligand. CD47 antibodies that are labeled appropriately are available commercially for this use (for example, the antibody product of clone B6H12 is available from Santa Cruz Biotechnology). The cells examined for CD47 phenotype can include standard tumour biopsy samples including particularly blood samples taken from the subject suspected of harbouring endogenous CD47+ cancer cells. CD47 disease cells of particular interest as targets for therapy with the present fusion proteins are those that “over-express” CD47. These CD47+ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry as exemplified herein or by immunostaining or by gene expression analysis or the like, to be greater than the level measurable on a counterpart cell having a CD47 phenotype that is normal for that cell type.

The present agent combination comprises both a CD47 blocking agent that preferably comprises a CD47-binding form of a SIRPα, as just described, and a taxane. In a preferred embodiment, the taxane is paclitaxel.

Paclitaxel is a secondary metabolite that is extractable from the bark of the Pacific yew tree and used in the treatment of various cancers including head and neck, breast and ovarian cancers. It is also useful to promote revascularization, in the treatment of restenosis, and in the treatment of non-small cell lung cancer and AIDS-related Kaposi's sarcoma. It acts by arresting the microtubules of cells, thereby preventing normal cell division and causing a G2/M phase blockage. Paclitaxel has also been shown to activate macrophages and to reprogram macrophages from the M2 phenotype (anti-inflammatory; immunosuppressive properties) to the M1 phenotype (classically activated; pro-inflammatory and anti-tumor properties) in a TLR4-dependent manner, thus further potentially contributing to its anti-cancer activity. (See, e.g. Wanderley, C, et al, Cancer Research, 78 (20); 15 Oct. 2018, pages 5891-5900).

Despite its complex chemical structure, shown below, total synthetic production has been achieved:

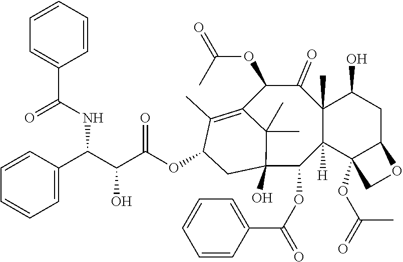 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

In IUPAC terms, paclitaxel is, (2α,4α,5β,7β,10β,13α)-4,10-bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate,

Paclitaxel is formulated for administration by infusion or by injection, yet is poorly soluble in water. Current paclitaxel formulations therefore use either non-aqueous solvents, such as DMSO, or they incorporate water-miscible solubilizers or lipid-based emulsions. In a marketed formulation, paclitaxel is mixed with Cremophor-EL (polyethoxylated castor oil) and ethanol, which transforms spontaneously into a microemulsion when diluted in sterile saline for administration.

The present invention is applicable also to other taxanes that are analogs of paclitaxel and retain its desirable cytotoxicity. In one embodiment, the taxane is a paclitaxel analog known as docetaxel, marketed as Taxotere®, and having the structure shown below:

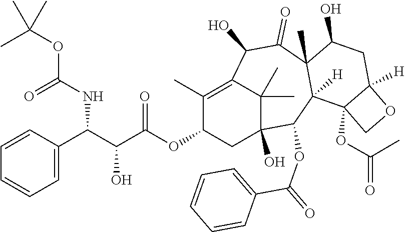

In another embodiment, the taxane is a pharmaceutically acceptable salt or ester of paclitaxel, including paclitaxel succinate. Another taxane that can be used in combination with CD47 blocking agent is Larotaxel, which is a semi-synthetic taxane having the structure (2α,3ξ, 4α,5β,7α,10β,13α)-4,10-bis(acetyloxy)-13-({(2R,3S)-3-[(tert-butoxycarbonyl)amino]-2-hydroxy-3-phenylpropanoyl}oxy)-1-hydroxy-9-oxo-5,20-epoxy-7,19-cyclotax-11-en-2-yl benzoate. Also useful in the present combination are taxane-related compounds that are epothilones, such as Ixabepilone.

Each agent included in the combination can be formulated separately for use in combination. The agents are said to be used “in combination” when, in a recipient of both agents, the effect of one agent enhances the effect of the other.

In this approach, each agent is provided in a dosage form comprising a pharmaceutically acceptable carrier, and in a therapeutically effective amount. As used herein, “pharmaceutically acceptable carrier” means any and all solvents, dispersion media, coatings, antibacterial and antifungal agents, isotonic and absorption delaying agents, and the like that are physiologically compatible and useful in the art of protein/antibody formulation. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent. Each of the SIRPαFc fusion protein and the taxane is formulated using practices standard in the art of therapeutic protein formulation. Solutions that are suitable for intravenous administration, such as by injection or infusion, are particularly useful. The taxane will of course be formulated as required by its insolubility and as permitted by the regulatory agencies that have approved its use in humans.

Sterile solutions can be prepared by incorporating the active compound in the required amount in an appropriate solvent with one or a combination of ingredients noted above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation are vacuum drying and freeze-drying (lyophilization) that yield a powder of the active ingredient plus any additional desired ingredient from a previously sterile-filtered solution thereof.

As used herein, “effective amount” and “treatment-effective” refer to an amounts effective, at dosages and for a particular period of time necessary, to achieve the desired therapeutic result. A therapeutically effective amount of each agent in the combination may vary according to factors such as the disease state, age, sex, and weight of the individual, and the ability of the agent to elicit a desired response in the recipient. A therapeutically effective amount is also one in which any toxic or detrimental effects of the pharmacological agent are outweighed by the therapeutically beneficial effects. The taxane will of course be formulated in amounts that are suitable for patient dosing, as permitted by the regulatory agencies that have approved its use in humans. For paclitaxel, effective doses will include 2-4 milligrams given intravenously such as by infusion over the course of 5-15 minutes, for instance.

The SIRPαFc fusion protein can be administered to the subject through any of the routes established for protein delivery, in particular intravenous, intradermal and subcutaneous injection or infusion, intratumoural injection, or by oral or nasal or pulmonary administration.

The agents in the present combination can be administered sequentially or, essentially at the same time. In embodiments, the taxane is given before administration of SIRPαFc. It is not essential that the taxane is present in a patient's system when the CD47 blocking agent is administered, although this is suitable. Thus in one embodiment there is provided a method for treating a subject presenting with CD47+ disease cells, comprising administering paclitaxel to the subject and then administering SIRPαFc to that subject in amounts sufficient to reduce the CD47+ disease/cancer cell population. In another embodiment there is provided a method for treating a subject presenting with CD47+ disease cells, comprising administering SIRPαFc to the subject and then administering paclitaxel to that subject in amounts sufficient to reduce the CD47+ disease/cancer cell population.

Dosing regimens are adjusted to provide the optimum desired response (e.g., a therapeutic response). For example, a single bolus of each agent may be administered, or several divided doses may be administered over time or the dose may be proportionally reduced or increased as indicated by the therapeutic situation. It is especially advantageous to formulate parenteral compositions in unit dosage form for ease of administration and uniformity of dosage. “Unit dosage form” as used herein refers to physically discrete units suited as unitary dosages for the subjects to be treated; each unit contains a predetermined quantity of active compound calculated to produce the desired therapeutic effect in association with the required pharmaceutical carrier.

The agents can be formulated in combination, so that the combination can be introduced to the recipient in one administration, e.g., one injection or one infusion bag. Alternatively, the agents can be combined as separate units that are provided together in a single package, and with instructions for the use thereof according to the present method. In another embodiment, an article of manufacture containing the SIRPαFc agent and taxane combination in an amount useful for the treatment of the disorders described herein is provided. The article of manufacture comprises one or both agents of the present antibody agent combination, as well as a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on or associated with the container indicates that the composition is used in combination with another CD47 blocking agent in accordance with the present invention, thereby to elicit a synergistic effect on the CD47+ disease cells. The article of manufacture may further comprise a second container comprising a pharmaceutically-acceptable buffer, such as phosphate-buffered saline, Ringer's solution and dextrose solution. It may further include other matters desirable from a commercial and use standpoint, including other buffers, diluents, filters, needles, syringes, and package inserts with instructions for use.

For administration, when SIRPαFc of SEQ ID NO: 3 or SEQ ID NO: 9 are used for instance, the suitable dose will be within the range from about 0.0001 to 100 mg/kg, and more usually 0.01 to 50 mg/kg, of the host body weight. For example, dosages can be 0.3 mg/kg body weight, 1 mg/kg body weight, 3 mg/kg body weight, 5 mg/kg body weight or 10 mg/kg body weight, 15 mg/kg body weight, 20 mg/kg body weight, or within the range of 0.1-25 mg/kg.

The SIRPαFc protein displays negligible binding to red blood cells, relative to most CD47 antibodies. There is accordingly no need to account for an RBC “sink” when dosing with the agent combination. Relative to the other CD47 blocking agents that are bound by RBCs, it is estimated that the present SIRPαFc fusion can be effective at doses that are less than half the doses required for agents that become RBC-bound, such as CD47 antibodies. Moreover, the SIRPα-Fc fusion protein is a dedicated antagonist of the SIRPα-mediated signal, as it displays negligible CD47 agonism. There is accordingly no need, when establishing medically useful unit dosing regimens, to account for any CD47 stimulation induced by the agent.

The agent combination is useful to treat a variety of CD47+ disease cells. These include particularly CD47+ cancer cells, including liquid and solid tumours. Solid tumours can be treated with the present agent combination, to reduce the size, number or growth rate thereof and to control growth of cancer stem cells. Such solid tumours include CD47+ tumours in bladder, brain, breast, lung, colon, ovary, prostate, liver, kidney and other tissues as well. In another embodiment, the agent combination can used to inhibit the growth or proliferation of hematological cancers. As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML) including P53-mutated AML; chronic lymphocytic leukemia (CLL); chronic myelogenous leukemia (CML); myeloproliferative disorder/neoplasm (MPDS); and myelodysplastic syndrome. “Lymphoma” includes T cell lymphomas, and may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma.

In some embodiments, the hematological cancer treated with the agent combination is a CD47+ leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer treated with the agent combination is a CD47+ lymphoma or myeloma selected from Hodgkin's lymphoma, both indolent and aggressive non-Hodgkin's lymphoma, Burkitt's lymphoma, follicular lymphoma (small cell and large cell), multiple myeloma (MM), giant cell myeloma, heavy-chain myeloma, and light chain or Bence-Jones myeloma as well as leiomyosarcoma.

The combination therapy, comprising CD47 blockade and taxane-mediated treatment, can also be exploited together with any other agent or modality useful in the treatment of the targeted indication, such as surgery as in adjuvant therapy, or with additional chemotherapy as in neoadjuvant therapy.

In some embodiments, a combination therapy provided herein comprising a CD47 blockade agent and a taxane does not also include a TLR agonist, such as a TLR4 agonist. In some embodiments provided herein is a combination therapy comprising a SIRPαFc fusion protein and paclitaxel, wherein the combination therapy does not include a TLR agonist (for example, a TLR4 agonist).

Incorporated by reference herein for all purposes is the content of U.S. Provisional Patent Application No. 63/273,851 (filed Oct. 29, 2021).

### EXAMPLES

### Example 1

### Paclitaxel Increases Macrophage SIRPαFc Induced Phagocytosis of Target Cells.

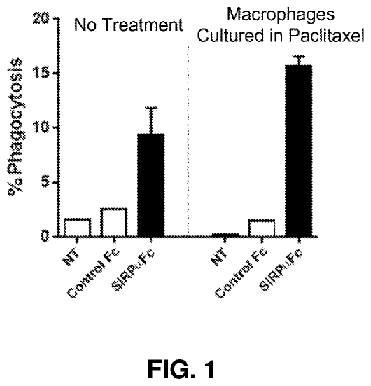

Heparinized whole blood was obtained from normal healthy human donors (Biological Specialty Corporation) and informed consent was obtained from all donors. Peripheral blood mononuclear cells (PBMCs) were isolated over Ficoll-Paque Plus density gradient (GE Healthcare) and CD14+ monocytes were isolated from PBMCs by positive selection using CD14 antibody-coated MicroBead separation (Miltenyi Biotec). Monocytes were differentiated into macrophages by culturing for seven days in X-Vivo-15 media (Lonza) supplemented with M-CSF (PeproTech), at which point paclitaxel was added to the macrophage culture for an additional six days. On the day of the phagocytosis assay, macrophages were co-cultured with violet proliferation dye 450 (VPD450)-labeled human B cell lymphoma cell line (Toledo) in the presence of 1 μM human SIRPαFc (IgV domain of human SIRPα variant 2 fused with IgG1 Fc), 1 μM control Fc [human IgG1 Fc region (hinge-CH2-CH3)] or nothing (no treatment, NT) for two hours. Phagocytosis was assessed as % VPD450+ cells of live, single CD14+CD11b+ macrophages by flow cytometry. Results are shown in FIG. 1 and are duplicates from a single experiment.

As shown in FIG. 1, substantial phagocytosis of the VPD450-labeled B cell lymphoma cells occurred with the human SIRPαFc but not with the control Fc or no treatment assays. In addition, the combination of paclitaxel with SIRPαFc resulted in an over 50% increase in phagocytosis as compared to the assay with SIRPαFc alone. Specifically, in the “no treatment”+SIRPαFc assay, the % phagocytosis was around 9%; in contrast, in the “macrophages cultured in paclitaxel”+SIRPαFc assay, the % phagocytosis was around 15%.

### Example 2

### The Combination of Paclitaxel and TTI-621 or TTI-622 Increases Macrophage Phagocytosis of Target Cells.

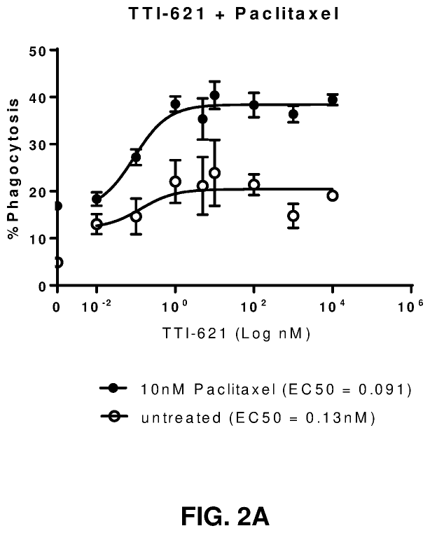

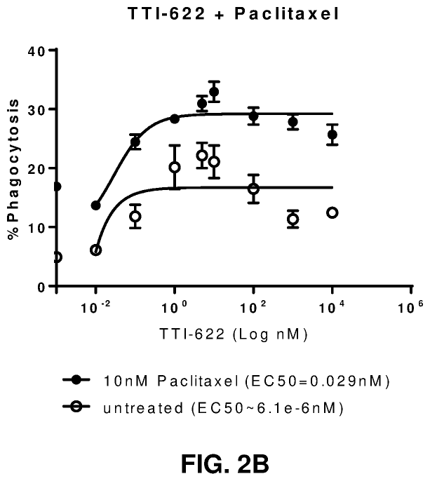

PBMC from normal donors were purchased from BioIVT and informed consent was obtained from all donors. CD14+ monocytes were isolated from PBMCs by positive selection using human monocyte isolation kit. Monocytes were differentiated into macrophages by culturing for at least ten days in X-Vivo-15 media (Lonza) supplemented with M-CSF (PeproTech), at which point Paclitaxel (Selleckchem) was added to the macrophage culture for an additional three days. Similarly, human ovarian cell line OVCAR-3 was also treated with Paclitaxel (Selleckchem) for three days prior to the phagocytosis assay. One day before the phagocytosis assay, macrophages were primed with IFNg (Pepro Tech). On the day of the phagocytosis assay, macrophages were co-cultured with violet proliferation dye 450 (VPD450)-human ovarian cell line OVCAR-3 in the presence of TTI-621 or TTI-622 for two hours. Phagocytosis was assessed as % VPD450+ cells of live, single CD14+CD11b+ macrophages by flow cytometry. Results are shown in FIGS. 2A and 2B.

As shown in FIG. 2A, the combination of TTI-621 with paclitaxel nearly doubled the % phagocytosis of the OVCAR-3 cells across most TTI-621 concentrations as compared to the % phagocytosis with TTI-621 alone. Similarly, as shown in FIG. 2B, the combination of TTI-622 with paclitaxel nearly doubled the % phagocytosis of the OVCAR-3 cells across most TTI-622 concentrations as compared to the % phagocytosis with TTI-622 alone.

While materials and methods of this disclosure have been described in terms of preferred embodiments, it will be apparent that variations may be applied to the articles and methods without departing from the spirit and scope of the disclosure. All such variations and equivalents, whether now existing or later developed, are deemed to be within the spirit and scope of the disclosure herein.

The disclosure illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed, and such modifications and variations are considered to be within the scope of this disclosure.

For conciseness, several aspects or embodiments are described herein as genera and/or as lists of alternative species. In each instance, subgenera and individual species are contemplated as individual aspects or embodiments of the invention. For aspects described as ranges, integer and half-integer subranges are specifically contemplated.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law). All headings and sub-headings are used herein for convenience only and should not be construed as being limiting in any way. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure. The citation and incorporation of patent documents herein is done for convenience only and does not reflect any view of the validity, patentability, and/or enforceability of such patent documents.

1. A method for treating a subject presenting with CD47⁺ cancer cells, comprising administering to the subject a CD47 blocking agent, and a taxane.

2. (canceled)

3. The method according to claim 1, wherein the taxane is selected from docetaxel and paclitaxel.

4. The method according to claim 3, wherein the taxane is paclitaxel.

5. The method according to claim 1, wherein the CD47 blocking agent comprises a CD47-binding form of human SIRPα.

6. The method according to claim 5, wherein the CD47-binding form of human SIRPα is a CD47-binding fragment of human SIRPα.

7. The method according to claim 6, wherein the CD47 binding fragment of human SIRPα comprises the IgV domain of human SIRPα.

8. The method according to claim 5, wherein the CD47 blocking agent comprises the CD47-binding form of human SIRPα attached to a heterologous peptide or protein.

9. The method according to claim 1, wherein the CD47 blocking agent is an Fc fusion protein comprising the IgV domain of soluble human SIRPα variant 2 attached to an antibody constant region (Fc).

10. The method according to claim 9, wherein the Fc fusion protein comprising soluble SIRPα comprises SEQ ID NO: 3.

11. The method according to claim 9, wherein the Fc fusion protein comprising soluble SIRPα comprises SEQ ID NO: 9.

12. The method according to claim 1, wherein the subject presents with CD47⁺ cancer cells that are blood cancer cells or solid tumour cells.

13. The method according to claim 12, wherein the subject presents with CD47⁺ cancer cells that are blood cancer cells.

14. The method according to claim 13, wherein the blood cancer cells are leukemia cells or lymphoma cells.

15. The method according to claim 12, wherein the cancer cells comprise solid tumour cells.

16. The method according to claim 15, wherein the solid tumour cancer cells are breast cancer cells, lung cancer cells, or ovarian cancer cells.

17. A combination of anti-cancer agents, comprising an amount of a CD47 blocking agent effective to deplete CD47⁺ cancer cells, and an amount of paclitaxel effective to enhance depletion of CD47⁺ cancer cells, together with instructions teaching the use thereof according to claim 1.

18. The use of the combination according to claim 17, for the treatment of a subject presenting with CD47⁺ disease cells.

19. The use according to claim 18, wherein the CD47⁺ disease cells are CD47+ cancer cells.

20. The use according to claim 19, wherein the CD47⁺ cancer cells comprise blood cancer cells or solid tumour cells.

21. A kit comprising unit dose formulations of a CD47 blockade drug and a taxane.

22. (canceled)


SEQUENCE LISTING (9 sequences)

SEQ ID NO: 1 - protein, 106 aa, synthetic construct
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 5 aa, synthetic construct
GGGGS

SEQ ID NO: 5 - protein, 11 aa, synthetic construct
GTELSVRAKPS

SEQ ID NO: 6 - protein, 118 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 7 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 347 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK